(19) United States
(12) Patent Application Publication
(10) Pub. No.: US 2023/0165504 A1
(43) Pub. Date: Jun. 1, 2023

(54) ELECTROCARDIOGRAM CONVERTING DEVICE

(71) Applicant: QT Medical, Inc., Taipei City (TW)

(72) Inventors: Jui-Chung CHANG, Taipei City (TW); Yu-Ting CHEN, Taipei City (TW); Ying-Lung CHENG, Taipei City (TW)

(21) Appl. No.: 18/059,987

(22) Filed: Nov. 30, 2022

(30) Foreign Application Priority Data
Nov. 30, 2021 (TW) 110144676

(51) Int. Cl. A61B 5/308

(52) U.S. Cl. CPC A61B 5/308; A61B 2562/227; A61B 2562/226

(57) ABSTRACT

An electrocardiogram converting device includes a casing, a first connector, a chest lead connection module, and a limb lead connection module. The casing includes a first surface, a second surface, and a third surface. The first connector is installed on the first surface of the casing, the chest lead connection module is installed on the second surface of the casing, and the limb lead connection module is installed on the third surface of the casing. In addition, the second surface is perpendicular to the third surface, and the first surface is parallel to the third surface, so as to reduce a length and a volume of the electrocardiogram converting device and improve the convenience of electrocardiogram measurement.

### RELATED APPLICATIONS

This application claims priority to Taiwan application Serial No. 110144676, filed Nov. 30, 2021, the disclosures of which are incorporated herein by reference in their entireties.

### BACKGROUND

### Technical Field

The present disclosure relates to an electrocardiogram converting device. More particularly, the present disclosure relates to a small electrocardiogram converting device.

### Description of Related Art

With the advancement of science and technology and the advancement of medical technology, the life span of human beings is also gradually increasing. At present, the world has gradually entered an aging society, and the proportion of the elderly population has increased year by year, and the heart disease has also become one invisible killer for the elderly.

Arrhythmia is a very common symptom in heart-related diseases, so the monitoring of heart-related diseases is an important issue for the elderly. At present, the detection of heart rate or electrocardiogram (ECG) may be mainly monitored by electrocardiogram measuring equipment. A traditional measure method is to stick wet electrodes containing electrolytes on the chest as a connection between the skin and the equipment to measure the heart rate or the electrocardiogram.

However, with the advancement of science and technology, various electrocardiogram measurement devices have been gradually developed. Besides the traditional wet electrodes, new disposable electrodes and personalized electrodes designed according to different body sizes have also gradually produced. However, when using the new measuring electrode patch to measure the electrocardiogram, the electrocardiogram measurement instrument may be located on the right side or left side of the person to be measured according to the site limitation or the needs of other instruments. Therefore, there is a need to conveniently and quickly connect the electrocardiogram cable and the electrode patch.

### SUMMARY

One objective of the embodiments of the present invention is to provide an electrocardiogram converting device to conveniently connect to an integrated electrode patch and an electrocardiogram (ECG) cable and improve measuring accuracy.

To achieve these and other advantages and in accordance with the objective of the embodiments of the present invention, as the embodiment broadly describes herein, the embodiments of the present invention provides an electrocardiogram converting device. The electrocardiogram converting device includes a casing, a first connector, a chest lead connection module and a limb lead connection module. The casing includes a first surface, a second surface and a third surface. The first connector is installed on the first surface of the casing, the chest lead connection module is installed on the second surface of the casing and the limb lead connection module is installed on the third surface of the casing. In addition, the second surface is perpendicular to the third surface, and the first surface is parallel with the third surface.

In some embodiments, the first connector includes a rotational shaft to allow the first connector to rotate on the first surface of the casing.

In some embodiments, the electrocardiogram converting device further includes a rotation angle limiter, a first rotation angle limiting partition and a second rotation angle limiting partition. The rotation angle limiter is connected to the rotational shaft. The first rotation angle limiting partition and the second rotation angle limiting partition are disposed inside the casing to limit a rotating angle of the rotation angle limiter.

In some embodiments, the rotating angle of the rotation angle limiter is about 180 degrees.

In some embodiments, the electrocardiogram converting device further includes insulation partitions disposed on the second surface and the third surface of the casing, and the chest lead connection module and the limb lead connection module are disposed on inner surfaces of the insulation partitions.

In some embodiments, the chest lead connection module and the limb lead connection module respectively include a plurality of banana sockets, and the banana sockets are aligned with corresponding openings of the insulation partitions.

In some embodiments, insulation handles of the banana connectors are inserted into the corresponding openings of the insulation partitions to shelter metal terminals of the banana connectors when the banana connectors are inserted into corresponding banana sockets.

In some embodiments, the chest lead connection module includes V1, V2, V3, V4, V5 and V6 electrocardiogram (ECG) signal connectors.

In some embodiments, the limb lead connection module includes LL, RL, LA and RA ECG signal connectors.

In some embodiments, the V1 ECG signal connector includes a red recognition ring, the V2 ECG signal connector includes a yellow recognition ring, the V3 ECG signal connector includes a green recognition ring, the V4 ECG signal connector includes a blue recognition ring, the V5 ECG signal connector includes an orange recognition ring, the V6 ECG signal connector includes a purple recognition ring, the LL ECG signal connector includes a red recognition ring, the RL ECG signal connector includes a green recognition ring, the LA ECG signal connector includes a black recognition ring, and the RA ECG signal connector includes a white recognition ring.

In some embodiments, the V1 ECG signal connector further includes a brown recognition ring surrounding the red recognition ring of the V1 ECG signal connector, the V2 ECG signal connector further includes a brown recognition ring surrounding the yellow recognition ring of the V2 ECG signal connector, the V3 ECG signal connector further includes a brown recognition ring surrounding the green recognition ring of the V3 ECG signal connector, the V4 ECG signal connector further includes a brown recognition ring surrounding the blue recognition ring of the V4 ECG signal connector, the V5 ECG signal connector further includes a brown recognition ring surrounding the orange recognition ring of the V5 ECG signal connector, and the V6 ECG signal connector further includes a brown recognition ring surrounding the purple recognition ring of the V6 ECG signal connector.

In some embodiments, the chest lead connection module includes C1, C2, C3, C4, C5 and C6 ECG signal connectors, and the limb lead connection module includes F, N, L and R ECG signal connectors.

In some embodiments, the C1 ECG signal connector includes a red recognition ring, the C2 ECG signal connector includes a yellow recognition ring, the C3 ECG signal connector includes a green recognition ring, the C4 ECG signal connector includes a brown recognition ring, the C5 ECG signal connector includes a black recognition ring, the C6 ECG signal connector includes a purple recognition ring, the F ECG signal connector includes a green recognition ring, the N ECG signal connector includes a black recognition ring, the L ECG signal connector includes a yellow recognition ring, and the R ECG signal connector includes a red recognition ring.

In some embodiments, the C1 ECG signal connector further includes a white recognition ring surrounding the red recognition ring, the C2 ECG signal connector further includes a white recognition ring surrounding the yellow recognition ring, the C3 ECG signal connector further includes a white recognition ring surrounding the green recognition ring, the C4 ECG signal connector further includes a white recognition ring surrounding the brown recognition ring, the C5 ECG signal connector further includes a white recognition ring surrounding the black recognition ring, and the C6 ECG signal connector further includes a white recognition ring surrounding the purple recognition ring.

In some embodiments, the first connector including a High-Definition Multimedia Interface (HDMI) connector with 29 pins, and connectors of the chest lead connection module are arranged staggered and connectors of the limb lead connection module are arranged staggered.

In some embodiments, the first connector is further connected an extension cable to electrically connect to an integrated electrode patch.

According to another aspect of the invention, an electrocardiogram converting device is provided. The electrocardiogram converting device includes a casing, a first connector, a chest lead connection module and a limb lead connection module. The casing includes a first surface, a second surface and a third surface, and the second surface is perpendicular to the third surface, and the first surface is parallel with the third surface. The first connector is fixed on the first surface of the casing, the chest lead connection module is installed on the second surface of the casing and the limb lead connection module is installed on the third surface of the casing.

In some embodiments, the first connector is accommodated in the casing.

In some embodiments, the first connector includes a High-Definition Multimedia Interface (HDMI) connector with 29 pins, and connectors of the chest lead connection module are arranged staggered and connectors of the limb lead connection module are arranged staggered.

According to further another aspect of the invention, an electrocardiogram converting device is provided. The electrocardiogram converting device includes a casing, a first connector, a chest lead connection module, a limb lead connection module and an extension cable. The casing includes a first surface, a second surface and a third surface, and the second surface is perpendicular to the third surface, and the first surface is parallel with the third surface. The first connector is fixed on the first surface of the casing and accommodated in the casing, and the first connector includes a High-Definition Multimedia Interface (HDMI) connector with 29 pins. The chest lead connection module is installed on the second surface of the casing. The limb lead connection module is installed on the third surface of the casing. In addition, the extension cable is connected to the first connector for connecting to an integrated electrode patch.

Hence, the electrocardiogram converting device can facilitate the measurement personnel to connect the ECG cable so as to improve the accuracy and comfort while measuring the electrocardiogram, and the convenience and practicability can be also improved with the color recognition rings of the electrocardiogram converting device. In addition, with the rotatable connector and/or the extension cable, the electrocardiogram measuring instrument can be placed in any position relative to the person to be measured so that the measuring accuracy and comfort may not be affected. In addition, the volume of the electrocardiogram converting device can be further reduced by arranging the position and angle of the chest lead connection module and the limb lead connection module.

### BRIEF DESCRIPTION OF THE DRAWINGS

The disclosure can be more fully understood by reading the following detailed description of the embodiment, with reference made to the accompanying drawings as follows:

FIG. 1 illustrates a schematic perspective diagram of an electrocardiogram converting device according to one embodiment of the present invention;

FIG. 2 illustrates a partial view of the schematic perspective diagram of the electrocardiogram converting device shown in FIG. 1;

FIG. 3 illustrates a cross-sectional view of the electrocardiogram converting device shown in FIG. 1;

FIG. 4 illustrates a reference diagram of the electrocardiogram converting device in an environment of use;

FIG. 5 illustrates a schematic perspective diagram of an electrocardiogram converting device according to another embodiment of the present invention;

FIG. 6 illustrates a schematic perspective diagram from another angle of the electrocardiogram converting device shown in FIG. 5;

FIG. 7 illustrates a schematic diagram shown the connections of the electrocardiogram converting device and banana connectors; and

FIG. 8 illustrates a schematic diagram of an extension cable according to one embodiment of the present invention.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

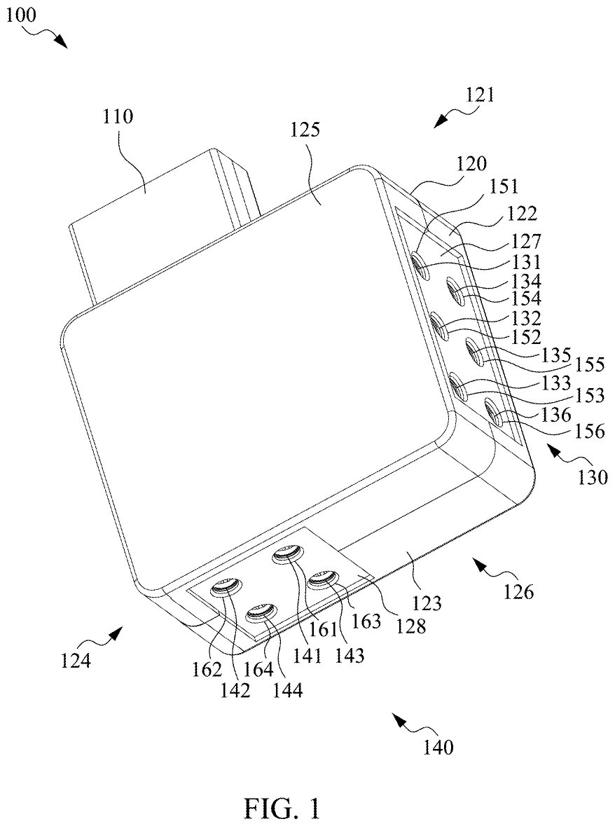

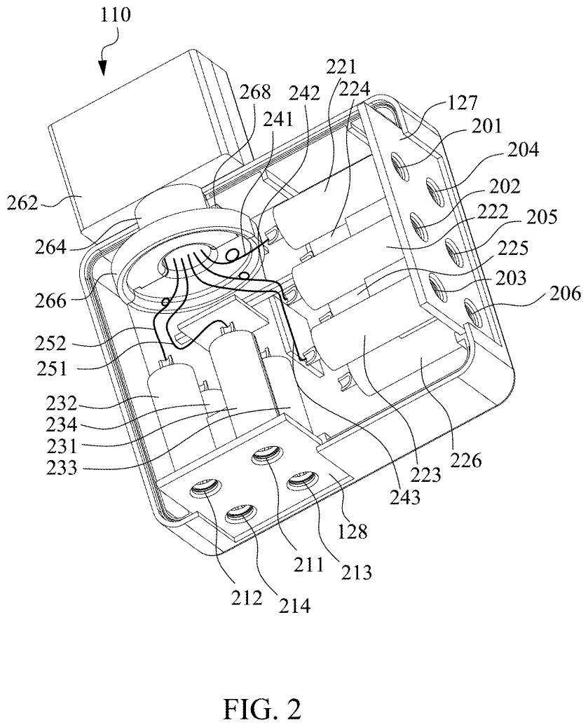

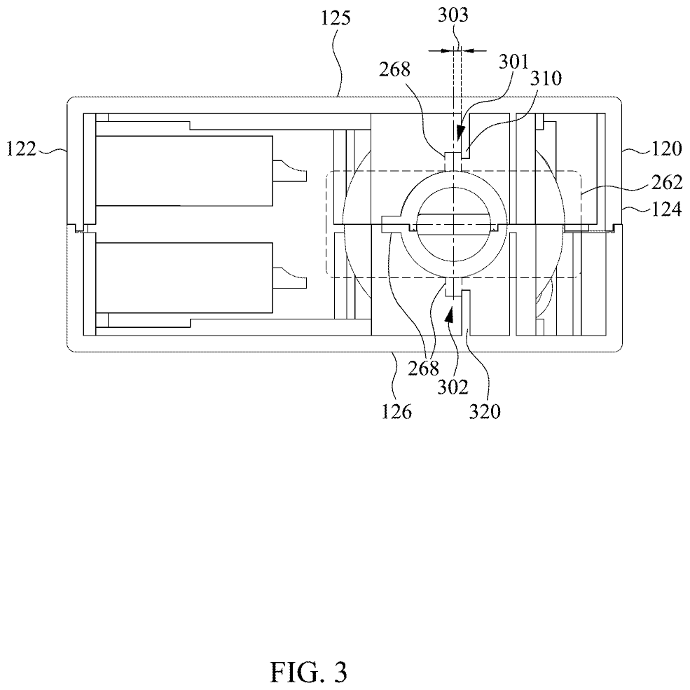

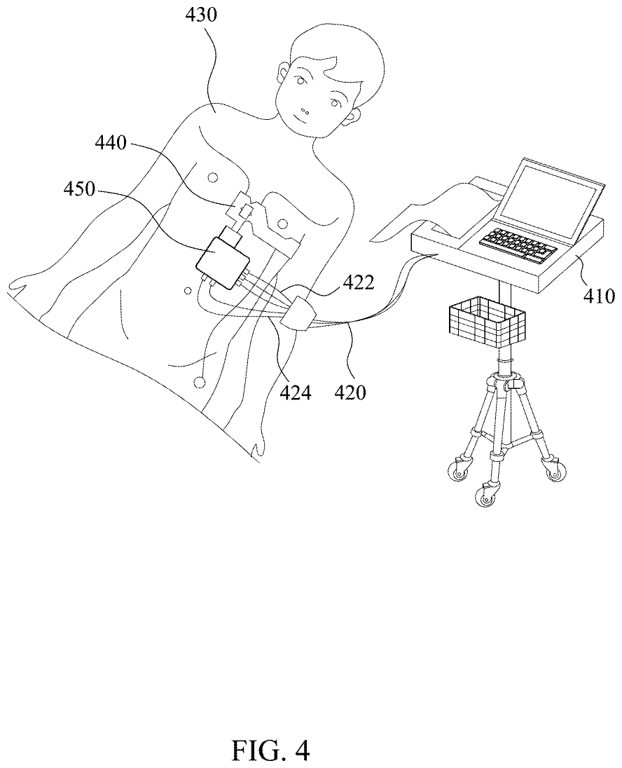

FIG. 1 illustrates a schematic perspective diagram of an electrocardiogram converting device according to one embodiment of the present invention, FIG. 2 illustrates a partial view thereof, FIG. 3 illustrates a cross-sectional view thereof, and FIG. 4 illustrates a reference diagram of the electrocardiogram converting device in an environment of use.

Referring to FIG. 1 and FIG. 2, the electrocardiogram converting device 100 includes a casing 120, a first connector 110, a chest lead connection module 130 and a limb lead connection module 140. The casing 120, such as a hexahedron casing, includes a first surface 121, a second surface 122, a third surface 123, a fourth surface 124, a fifth surface 125 and a sixth surface 126. The first surface 121 is opposite to the third surface 123, the second surface 122 is opposite to the fourth surface 124, and the fifth surface 125 is opposite to the sixth surface 126 and connects to the first surface 121, the second surface 122, the third surface 123 and the fourth surface 124.

In some embodiments, the casing 120 is a rectangular casing, and the second surface 122 is perpendicular to the first surface 121 and the third surface 123.

The first connector 110 is installed on the first surface 121 of the casing 120, and the chest lead connection module 130 is installed on the second surface 122 of the casing 120. In addition, the limb lead connection module 140 is installed on the third surface 123 of the casing 120. In addition, the second surface 122 is perpendicular to the third surface 123, and the first surface 121 is parallel with the third surface 123.

In some embodiments, the first connector 110 includes an external connector 262 to connect to an integrated electrode patch, a rotational shaft 264 and a wire gathering turntable 266. Therefore, the first connector 110 may rotate on the first surface 121 of the casing 120 through the rotational shaft 264.

Simultaneously referring to FIG. 3, in some embodiments, the first connector 110 further includes a rotation angle limiter 268 disposed on the rotational shaft 264. A first rotation angle limiting partition 310 and a second rotation angle limiting partition 320 are disposed on an inner surface of the casing 120. When the first connector 110 is rotated, the rotation angle limiter 268 may contact the first rotation angle limiting partition 310 or the second rotation angle limiting partition 320 to limit a rotating angle of the first connector 110 and the rotation angle limiter 268 relative to the casing 120. For example, when the rotation angle limiter 268 is clockwise rotated to a first position 301 shown in FIG. 3, the rotation angle limiter 268 contacts the first rotation angle limiting partition 310 and stops. In addition, the rotation angle limiter 268 may counterclockwise rotate to a second position 302 shown in FIG. 3, and the rotation angle limiter 268 may contact the second rotation angle limiting partition 320 to stop. Therefore, the external connector 262 may be rotatable relative to the casing 120 between an angle limited by the first rotation angle limiting partition 310 and the second rotation angle limiting partition 320.

In some embodiments, the rotating angle of the first connector 110 and the rotation angle limiter 268 relative to casing 120 is about 180 degrees.

In some embodiments, an eccentricity 303 is formed between a center of the rotational shaft 264 and the first rotation angle limiting partition 310, and a center of the rotational shaft 264 and the second rotation angle limiting partition 320, for example, the eccentricity 303 is about a half thickness of the rotation angle limiter 268 to allow the first connector 110 and the rotation angle limiter 268 to be precisely positioned at a predetermined position of the casing 120.

Simultaneously referring to FIG. 4, a person 430 to be measured uses the electrocardiogram measurement instrument 410 to measure the electrocardiogram. Generally speaking, the electrocardiogram measurement instrument 410 may locate at left side of the person 430 to be measured as illustrated in FIG. 4. However, the electrocardiogram measurement instrument 410 may be located on the right side or left side of the person 430 to be measured according to the site limitation or the needs of other measuring instruments.

When the electrocardiogram measurement instrument 410 is located on the right side of the person 430 to be measured, the electrocardiogram cable 420 has to be folded to connect to the electrocardiogram measurement instrument 410 because the traditional electrocardiogram converting device cannot be rotated. Therefore, the folded electrocardiogram cable not only causes discomfort to the person 430 to be measured, but also may affect the measurement results.

The electrocardiogram converting device 450 may conveniently rotate the chest lead connection module so the the chest lead connection module may be rotated from the left side of the person 430 to be measured to the right side of the person 430 to be measured. Therefore, the electrocardiogram measurement instrument 410 may located on the left side or the right side of the person 430 to be measured to accurately measure the electrocardiogram data.

Therefore, a measurement personnel only need to rotate the electrocardiogram converting device 450 to a suitable direction and connect the integrated electrode patch 440, then the chest lead cable 422 and the limb lead cable 424 of the electrocardiogram cable ECG cable 420 can be respectively inserted into corresponding connectors, and the electrocardiogram measurement can be accurately performed without affecting the person 430 to be measured. In addition, the measurement personnel can also perform the electrocardiogram measurement at any time by adjusting the required direction of the electrocardiogram converting device 450 according to the position of the electrocardiogram measurement instrument 410 relative to the person 430 to be measured, even after the electrocardiogram cable ECG cable 420 is connected to the electrocardiogram measurement instrument 410, the direction of the electrocardiogram converting device 450 can be adjusted in situ and the electrocardiogram data can still be measured without affecting the person 430 to be measured, so as to carry out the electrocardiogram measurement conveniently and accurately.

Referring to FIG. 2 again, in some embodiments, the electrocardiogram converting device 100 further includes an insulation partition 127 and an insulation partition 128 respectively disposed on the second surface 122 and the third surface 123 of the casing 120, and the chest lead connection module 130 and the limb lead connection module 140 are disposed on the inner surfaces of the insulation partition 127 and the insulation partition 128. In addition, the chest lead connection module 130 and the limb lead connection module 140 respectively include a plurality of banana sockets, and the banana sockets are respectively aligned with corresponding openings on the insulation partition 127 and the insulation partition 128, and the corresponding openings of the insulation partition 127 and the insulation partition 128 may shelter the metal elements of the banana connectors when the banana connectors are inserted into corresponding banana sockets.

In some embodiments, the banana sockets includes a first banana socket 221, a second banana socket 222, a third banana socket 223, a fourth banana socket 224, a fifth banana socket 225, a sixth banana socket 226, a seventh banana socket 231, an eighth banana socket 232, a ninth banana socket 233 and a tenth banana socket 234 respectively aligned with an opening 201, an opening 202, an opening 203, an opening 204, an opening 205, an opening 206, an opening 211, an opening 212, an opening 213 and an opening 214, and the metal terminals of the banana sockets are covered by the corresponding openings to prevent the metal terminals of the banana sockets from being exposed. In addition, the corresponding openings of the insulation partition 127 and the insulation partition 128 can also shelter the metal portions of the banana connectors to prevent the metal portion of the banana connectors from being exposed so as to avoid affecting the measure signals and improve the measurement accuracy when the banana connectors are inserted into the corresponding banana sockets.

In some embodiments, the insulation partition 127 and the insulation partition 128 may be integrally formed with the casing 120 without departing from the scope or spirit of the disclosure.

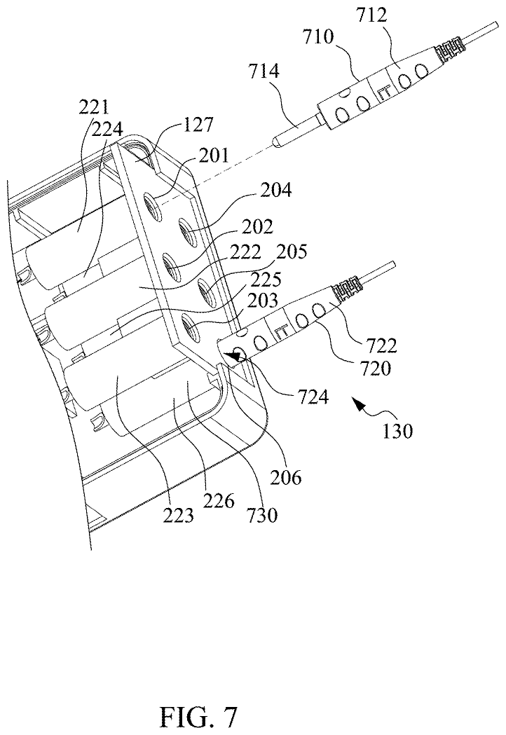

In some embodiments, referring to FIG. 7, the chest lead connection module 130 and the insulation partition 127 are illustrated as an example. The chest lead connection module 130 is disposed on the inner surface of the insulation partition 127, the chest lead connection module 130 includes a plurality of banana sockets, and the banana sockets are aligned with corresponding openings of the insulation partition 127.

In addition, the banana connector 710 and the banana connector 720 are illustrated as an example. The banana connector 710 includes a metal terminal 714 and an insulation handle 712, and the banana connector 720 includes a metal terminal 724 and an insulation handle 722. As shown in FIG. 7, the banana connector 710 is not inserted into the opening 201, and the banana connector 720 is inserted into the opening 206 to electrically connect to the sixth banana socket 226. In addition, the sixth banana socket 226 is illustrated as an example. The sixth banana socket 226 includes a metal tube 730, and the metal tube 730 is hidden inside the casing 120 and the insulation partition 127 to prevent from being exposed outside the casing 120 and the insulation partition 127.

In some embodiments, when the banana connector 720 is inserted into the opening 206 to electrically connect to the sixth banana socket 226, the insulation handle 722 of the banana connector 720 is preferably inserted into the opening 206 of the insulation handle 722, and the metal terminal 724 of the banana connector 720 is electrically connected to the metal tube 730 of the sixth banana socket 226 to prevent the metal terminal 724 and the metal tube 730 of the sixth banana socket 226 being exposed outside the casing 120 and the insulation partition 127 so as to improve the measurement accuracy and stability and avoid external signal interference and prevent the measuring signals from interfering the other electronic instruments. In some embodiments, but not limited to this, the metal terminal 724 of the banana connector 720 may be inserted into the opening 206 to electrically connect to the metal tube 730 of the sixth banana socket 226, and the insulation handle 722 of the banana connector 720 covers on the insulation partition 127 to shelter the metal terminal 724 of the banana connector 720, without departing from the scope or spirit of the disclosure.

In addition, according to some embodiments of the electrocardiogram converting device 100, the chest lead connection module 130 is installed on the second surface 122 of the casing 120, and the limb lead connection module 140 is installed on the third surface 123 of the casing 120, and the second surface 122 is perpendicular to the third surface 123. Therefore, the length and volume of the electrocardiogram converting device may be efficiently reduced, and the lengths of the connecting wires may be almost the same with each other to improve the measurement accuracy and facilitate the operation of the measurement personnel. As shown in FIG. 2, the distances between the wire gathering turntable 266 of the first connector 110 and the ends of the first banana socket 221, the second banana socket 222, the third banana socket 223, the fourth banana socket 224, the fifth banana socket 225, the sixth banana socket 226, the seventh banana socket 231, the eighth banana socket 232, the ninth banana socket 233 and the banana sockets tenth banana socket 234 are almost the same with each other, and the distances between the wire gathering turntable and the banana sockets are reduced to reduce the lengths of the connecting wires so as to effectively reduce the variation of signal transmission. For example, the first connecting wire 241, the second connecting wire 242, the third connecting wire 243, the seventh connecting wire 251 and the eighth connecting wire 252 are illustrated as an example. The lengths of the connecting wires are configured to have the same length and the maximum length of the connecting wires is also reduced. The uniform connecting wires are electrically connected the banana sockets and the first connector 110 to further improve the measurement accuracy of the electrocardiogram converting device 100.

In some embodiments, the chest lead connection module 130 includes a first chest lead connector 131, a second chest lead connector 132, a third chest lead connector 133, a fourth chest lead connector 134, a fifth chest lead connector 135 and a sixth chest lead connector 136, for example, a V1 ECG signal connector, a V2 ECG signal connector, a V3 ECG signal connector, a V4 ECG signal connector, a V5 ECG signal connector and a V6 ECG signal connector, but not limited thereto.

In addition, the limb lead connection module 140 includes a first limb lead connector 141, a second limb lead connector 142, a third limb lead connector 143 and a fourth limb lead connector 144, for example, a LL ECG signal connector, a RL ECG signal connector, a LA ECG signal connector and a RA ECG signal connector, but not limited thereto.

In some embodiments, in order to facilitate the measurement personnel to connect the electrocardiogram cable ECG cable and the electrocardiogram converting device 100, the electrocardiogram converting device 100 is further equipped with color recognition rings 151, 152, 153, 154, 155, 156 and color recognition rings 161, 162, 163, 164. For example, the V1 ECG signal connector has a red recognition ring, the V2 ECG signal connector has a yellow recognition ring, the V3 ECG signal connector has a green recognition ring, the V4 ECG signal connector has a blue recognition ring, the V5 ECG signal connector has an orange recognition ring, the V6 ECG signal connector has a purple recognition ring, the LL ECG signal connector has a red recognition ring, the RL ECG signal connector has a green recognition ring, the LA ECG signal connector has a black recognition ring, and the RA ECG signal connector has a white recognition ring, but not limited thereto.

In some embodiments, the color recognition rings 151, 152, 153, 154, 155, 156 may be two-color recognition rings, for example, the V1 ECG signal connector has a brown recognition ring surrounding the red recognition ring, the V2 ECG signal connector has a brown recognition ring surrounding the yellow recognition ring, the V3 ECG signal connector has a brown recognition ring surrounding the green recognition ring, the V4 ECG signal connector has a brown recognition ring surrounding the blue recognition ring, the V5 ECG signal connector has a brown recognition ring surrounding the orange recognition ring, and the V6 ECG signal connector has a brown recognition ring surrounding the purple recognition ring.

In another embodiments, the chest lead connection module includes the C1, C2, C3, C4, C5 and C6 ECG signal connectors, and the limb lead connection module includes the F, N, L and R ECG signal connectors. The C1 ECG signal connector has the red recognition ring, the C2 ECG signal connector has the yellow recognition ring, the C3 ECG signal connector has the green recognition ring, the C4 ECG signal connector has the brown recognition ring, the C5 ECG signal connector has the black recognition ring, the C6 ECG signal connector has the purple recognition ring, the F ECG signal connector has the green recognition ring, the N ECG signal connector has the black recognition ring, the L ECG signal connector has the yellow recognition ring, and the R ECG signal connector has the red recognition ring.

In addition, the foregoing color recognition rings may be two-color recognition rings, for example, the C1 ECG signal connector has a white recognition ring surrounding the red recognition ring, the C2 ECG signal connector has a white recognition ring surrounding the yellow recognition ring, the C3 ECG signal connector has a white recognition ring surrounding the green recognition ring, the C4 ECG signal connector has a white recognition ring surrounding the brown recognition ring, the C5 ECG signal connector has a white recognition ring surrounding the black recognition ring, and the C6 ECG signal connector has a white recognition ring surrounding the purple recognition ring.

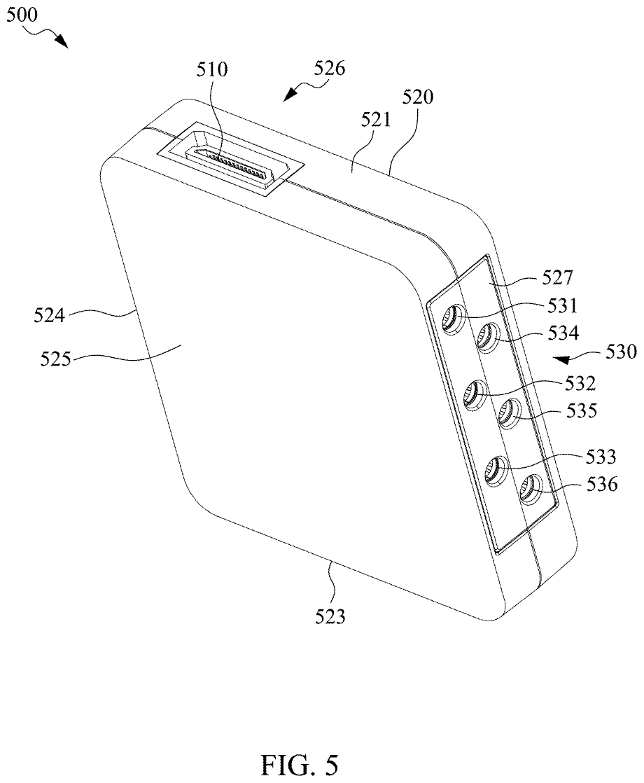

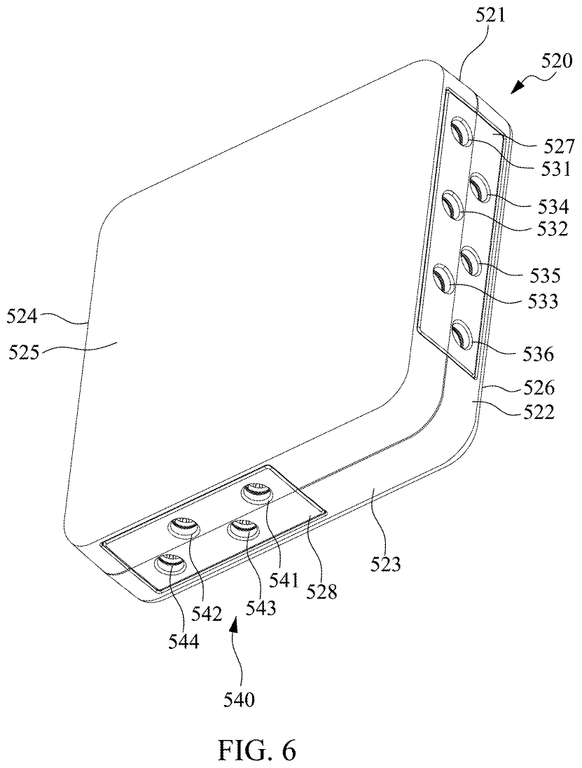

FIG. 5 illustrates a schematic perspective diagram of an electrocardiogram converting device according to another embodiment of the present invention, and FIG. 6 illustrates the electrocardiogram converting device from another angle. The electrocardiogram converting device 500 includes a casing 520, a first connector 510, a chest lead connection module 530 and a limb lead connection module 540. The casing 520, for example a hexahedron casing, includes a first surface 521, a second surface 522, a third surface 523, a fourth surface 524, a fifth surface 525 and a sixth surface 526. The first surface 521 is configured opposite to the third surface 523, the second surface 522 is configured opposite to the fourth surface 524, and the fifth surface 525 is configured opposite to the sixth surface 526. The fifth surface 525 and the sixth surface 526 connect to the first surface 521, the second surface 522, the third surface 523 and the fourth surface 524.

In some embodiments, the casing 520 may be a rectangular casing, and the second surface 522 is perpendicular to the first surface 521 and the third surface 523.

The first connector 510 is installed on the first surface 521 of the casing 520, and the chest lead connection module 530 is installed on the second surface 522 of the casing 520. In addition, the limb lead connection module 540 is installed on the third surface 523 of the casing 520. In addition, the second surface 522 is perpendicular to the third surface 523, and the first surface 521 is parallel with the third surface 523.

In some embodiments, the first connector 510 may be a High-Definition Multimedia Interface (HDMI) connector accommodated in the casing 520, such as a 29-pin HDMI connector.

In some embodiments, the chest lead connection module 530 includes a first chest lead connector 531, a second chest lead connector 532, a third chest lead connector 533, a fourth chest lead connector 534, a fifth chest lead connector 535 and a sixth chest lead connector 536, for example, the V1 ECG signal connector, the V2 ECG signal connector, the V3 ECG signal connector, the V4 ECG signal connector, the V5 ECG signal connector and the V6 ECG signal connector. In addition, the limb lead connection module 540 includes the first limb lead connector 541, the second limb lead connector 542, the third limb lead connector 543 and the fourth limb lead connector 544, for example, the LL ECG signal connector, the RL ECG signal connector, the LA ECG signal connector and the RA ECG signal connector, but not limited thereto. It is worth noting that the connectors of the chest lead connection module 530 and the limb lead connection module 540 are arranged staggered to further reduce the thickness of the electrocardiogram converting device 500. In addition, the connectors of the chest lead connection module 530 and the limb lead connection module 540 also include color recognition rings, without departing from the scope or spirit of the disclosure.

In some embodiments, the electrocardiogram converting device 500 further includes an insulation partition 527 and insulation partition 528 respectively disposed on the second surface 522 and the third surface 523 of casing 520, and the chest lead connection module 530 and the limb lead connection module 540 are disposed on the inner surfaces of the insulation partition 527 and the insulation partition 528. In addition, the chest lead connection module 530 and the limb lead connection module 540 respectively include a plurality of banana sockets, and the banana sockets are aligned with corresponding openings of the insulation partition 527 and the insulation partition 528, and the corresponding openings of the insulation partition 527 and the insulation partition 528 shelter the metal portions of the banana connectors when the banana connectors are inserted into the corresponding banana sockets.

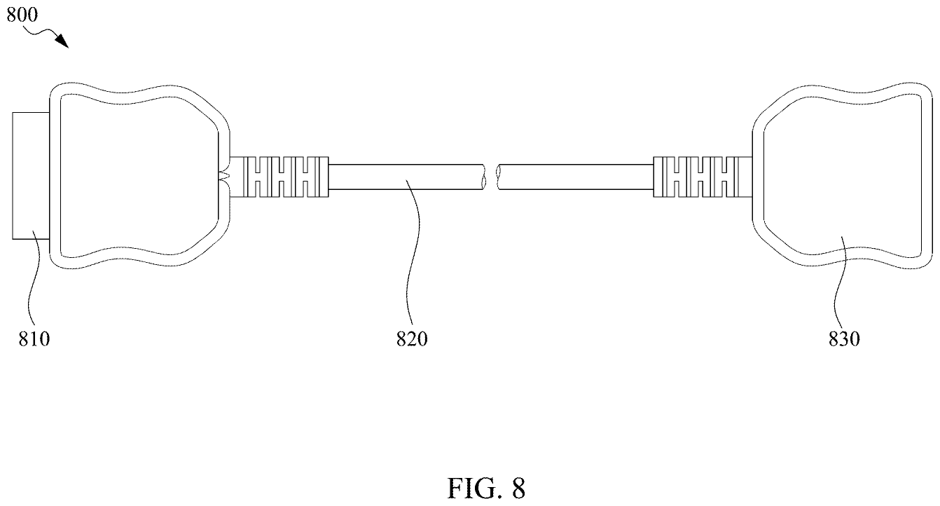

In addition, an extension cable according to one embodiment of the present invention is illustrated in FIG. 8. The extension cable 800 includes a first cable connector 810, a cable wire 820 and a second cable connector 830. Simultaneously referring to FIG. 5, the first cable connector 810 may connect to the first connector 510, and the second cable connector 830 may connect to the integrated electrode patch 440 (see FIG. 4) so as to facilitate the measurement personnel to electrically connect the electrocardiogram converting device to the integrated electrode patch 440, and also facilitate the measurement personnel to move the electrocardiogram measurement instrument 410.

Accordingly, the electrocardiogram converting device as mentioned above can facilitate the measurement personnel to connect the ECG cable so as to improve the accuracy and comfort while measuring the electrocardiogram, and the convenience and practicability can be also improved with the color recognition rings of the electrocardiogram converting device. In addition, with the rotatable connector and/or the extension cable, the electrocardiogram measuring instrument can be placed in any position relative to the person to be measured so that the measuring accuracy and comfort may not be affected. In addition, the volume of the electrocardiogram converting device can be further reduced by arranging the position and angle of the chest lead connection module and the limb lead connection module.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An electrocardiogram converting device, comprising:
a casing comprising a first surface, a second surface and a third surface;
a first connector installed on the first surface of the casing;
a chest lead connection module installed on the second surface of the casing; and
a limb lead connection module installed on the third surface of the casing, wherein the second surface is perpendicular to the third surface, and the first surface is parallel with the third surface.

2. The electrocardiogram converting device of claim 1, wherein the first connector comprises:
a rotational shaft to allow the first connector to rotate on the first surface of the casing.

3. The electrocardiogram converting device of claim 2, further comprising:
a rotation angle limiter connecting to the rotational shaft;
a first rotation angle limiting partition; and
a second rotation angle limiting partition, wherein the first rotation angle limiting partition and the second rotation angle limiting partition disposed inside the casing to limit a rotating angle of the rotation angle limiter.

4. The electrocardiogram converting device of claim 3, wherein the rotating angle of the rotation angle limiter is about 180 degrees.

5. The electrocardiogram converting device of claim 1, further comprising insulation partitions disposed on the second surface and the third surface of the casing, wherein the chest lead connection module and the limb lead connection module are disposed on inner surfaces of the insulation partitions.

6. The electrocardiogram converting device of claim 5, wherein the chest lead connection module and the limb lead connection module respectively comprise a plurality of banana sockets, and the banana sockets are aligned with corresponding openings of the insulation partitions.

7. The electrocardiogram converting device of claim 6, wherein insulation handles of banana connectors are inserted into the corresponding openings of the insulation partitions to shelter metal terminals of the banana connectors when the banana connectors are inserted into corresponding banana sockets.

8. The electrocardiogram converting device of claim 5, wherein the chest lead connection module comprises V1, V2, V3, V4, V5 and V6 electrocardiogram (ECG) signal connectors.

9. The electrocardiogram converting device of claim 8, wherein the limb lead connection module comprises LL, RL, LA and RA ECG signal connectors.

10. The electrocardiogram converting device of claim 9, wherein the V1 ECG signal connector comprises a red recognition ring, the V2 ECG signal connector comprises a yellow recognition ring, the V3 ECG signal connector comprises a green recognition ring, the V4 ECG signal connector comprises a blue recognition ring, the V5 ECG signal connector comprises an orange recognition ring, the V6 ECG signal connector comprises a purple recognition ring, the LL ECG signal connector comprises a red recognition ring, the RL ECG signal connector comprises a green recognition ring, the LA ECG signal connector comprises a black recognition ring, and the RA ECG signal connector comprises a white recognition ring.

11. The electrocardiogram converting device of claim 10, wherein the V1 ECG signal connector further comprises a brown recognition ring surrounding the red recognition ring of the V1 ECG signal connector, the V2 ECG signal connector further comprises a brown recognition ring surrounding the yellow recognition ring of the V2 ECG signal connector, the V3 ECG signal connector further comprises a brown recognition ring surrounding the green recognition ring of the V3 ECG signal connector, the V4 ECG signal connector further comprises a brown recognition ring surrounding the blue recognition ring of the V4 ECG signal connector, the V5 ECG signal connector further comprises a brown recognition ring surrounding the orange recognition ring of the V5 ECG signal connector, and the V6 ECG signal connector further comprises a brown recognition ring surrounding the purple recognition ring of the V6 ECG signal connector.

12. The electrocardiogram converting device of claim 5, wherein the chest lead connection module comprises C1, C2, C3, C4, C5 and C6 ECG signal connectors, and the limb lead connection module comprises F, N, L and R ECG signal connectors.

13. The electrocardiogram converting device of claim 12, wherein the C1 ECG signal connector comprises a red recognition ring, the C2 ECG signal connector comprises a yellow recognition ring, the C3 ECG signal connector comprises a green recognition ring, the C4 ECG signal connector comprises a brown recognition ring, the C5 ECG signal connector comprises a black recognition ring, the C6 ECG signal connector comprises a purple recognition ring, the F ECG signal connector comprises a green recognition ring, the N ECG signal connector comprises a black recognition ring, the L ECG signal connector comprises a yellow recognition ring, and the R ECG signal connector comprises a red recognition ring.

14. The electrocardiogram converting device of claim 13, wherein the C1 ECG signal connector further comprises a white recognition ring surrounding the red recognition ring, the C2 ECG signal connector further comprises a white recognition ring surrounding the yellow recognition ring, the C3 ECG signal connector further comprises a white recognition ring surrounding the green recognition ring, the C4 ECG signal connector further comprises a white recognition ring surrounding the brown recognition ring, the C5 ECG signal connector further comprises a white recognition ring surrounding the black recognition ring, and the C6 ECG signal connector further comprises a white recognition ring surrounding the purple recognition ring.

15. The electrocardiogram converting device of claim 1, wherein the first connector comprises a High-Definition Multimedia Interface (HDMI) connector with 29 pins, and connectors of the chest lead connection module are arranged staggered and connectors of the limb lead connection module are arranged staggered.

16. The electrocardiogram converting device of claim 1, wherein the first connector is further connected an extension cable to electrically connect to an integrated electrode patch.

17. An electrocardiogram converting device, comprising:
a casing comprising a first surface, a second surface and a third surface;
a first connector fixed on the first surface of the casing;
a chest lead connection module installed on the second surface of the casing; and
a limb lead connection module installed on the third surface of the casing, wherein the second surface is perpendicular to the third surface, and the first surface is parallel with the third surface.

18. The electrocardiogram converting device of claim 17, wherein the first connector is accommodated in the casing.

19. The electrocardiogram converting device of claim 18, wherein the first connector comprises a High-Definition Multimedia Interface (HDMI) connector with 29 pins, and connectors of the chest lead connection module are arranged staggered and connectors of the limb lead connection module are arranged staggered.

20. An electrocardiogram converting device, comprising:
a casing comprising a first surface, a second surface and a third surface, wherein the second surface is perpendicular to the third surface, and the first surface is parallel with the third surface;
a first connector fixed on the first surface of the casing and accommodated in the casing, wherein the first connector comprises a High-Definition Multimedia Interface (HDMI) connector with 29 pins;
a chest lead connection module installed on the second surface of the casing;
a limb lead connection module installed on the third surface of the casing; and
an extension cable connected to the first connector for connecting to an integrated electrode patch.
